(19) United States
(12) Patent Application Publication
(10) Pub. No.: US 2024/0317946 A1
(43) Pub. Date: Sep. 26, 2024

(54) MODIFIED COLLAGEN, PHOTO-CROSSLINKED BIOMATERIAL AND ITS PREPARATION METHOD AND APPLICATION

(71) Applicant: Wuzhou Victory Biotech Co., Ltd., Wuzhou City (CN)

(72) Inventors: CHEN-CHIH TSAI, Wuzhou City (CN); SONG-YI WU, Wuzhou City (CN); YA-XIAN ZHOU, Wuzhou City (CN)

(21) Appl. No.: 18/124,176

(22) Filed: Mar. 21, 2023

(51) Int. Cl. C08J 3/28; C07K 14/78; C08K 5/07; C08K 5/09; C08K 5/49

(52) U.S. Cl. CPC C08J 3/28; C07K 14/78; C08K 5/07; C08K 5/09; C08K 5/49

(57) ABSTRACT

In a modified collagen, photo-crosslinked biomaterial and its preparation method and application, the modified collagen includes a collagen grafted with a group represented by Formula (A). The photo-crosslinked biomaterial includes a hydrogel formed by curing the modified collagen. The photo-crosslinked biomaterial of the present invention is a biomaterial with good biocompatibility and cell compatibility, which has important application value in the following fields: repair and reconstruction of tissues and organs in vivo or in vitro, tissue engineering, 3D printing substrates and cell culture carriers, etc.

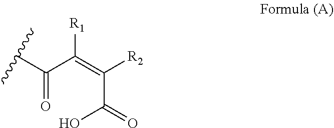

### FIELD OF THE INVENTION

The present invention relates to a modified collagen, photo-crosslinked biomaterial and its preparation method and application.

### BACKGROUND

Biomaterials are one of main components of living organisms. They have excellent biocompatibility and can play their functions of assisting and regulating biological tissues in vivo or in vitro. Among the components of human tissue, collagen is the main component of extracellular matrix. Its unique biological activity and three-dimensional structure play an important role in normal physiological functions of tissues, organs and damage repair, and therefore it has great potential in the field of Biomaterials.

At present, the main method of preparing hydrogels is performed by physical crosslinking method using a repeated freeze-thaw process, or by chemical crosslinking method with a crosslinking agent. However, the repeated freeze-thaw process is complicated, and the equipment requirements are high. In addition, the method using crosslinking agent raise concerns about toxic residues.

### SUMMARY OF INVENTION

In view of the aforementioned problems in the prior art, one of the objectives of the present invention is to provide a photo-crosslinked biomaterial with good biocompatibility and cell compatibility, which has important application value in the following fields: repair and reconstruction of tissues and organs in vivo or in vitro, tissue engineering, 3D printing substrates and cell culture carriers, etc. The present invention also provides a preparation method of the photo-crosslinked biomaterial, such method has the advantages of fast curing, simple process, and being not prone to toxic residues.

The present inventors found that if the crosslinking and curing process is carried out through ultraviolet light, and then no crosslinking agent is needed. The present method has the advantages of fast curing, simple process and is not prone to toxic residues, and can achieve in-situ crosslinking to solve the defects of traditional hydrogel preparation methods.

The present invention is as described below.

In a first aspect, the present invention provides a modified collagen, including a collagen grafted with a group represented by Formula (A).

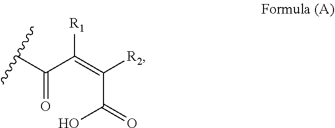

In Formula (A), R₁ and R₂ may be the same or different, and they are each independently selected from the group consisting of hydrogen, C₁-C₆ alkyl, C₁-C₆ alkoxy, C₆-C₁₀ aryl, or halogen. [symbol] represents the position where the group represented by Formula (A) connected to the collagen.

In some embodiments, the group represented by the Formula (A) is grafted on a free amino group of the collagen.

In some embodiments, the grafting rate of the modified collagen is at least 40%. For example, it can be 40%, 50%, 60%, 70%, 80%, 90%, 92%, 94%, 96%, 98%, 99% and any value in between. More than 90% is ideal, and more than 95% is more desirable.

In a second aspect, the present invention provides a method for preparing modified collagen, including carrying out a modification reaction between a collagen-containing raw material solution I and a raw material solution II with a compound of Formula (B),

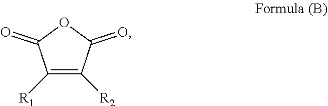

In Formula (B), R₁ and R₂ may be the same or different, and they are each independently selected from the group consisting of hydrogen, C₁-C₆ alkyl, C₁-C₆ alkoxy, C₆-C₁₀ aryl, or halogen.

In some embodiments, R₁ and R₂ in formula (B) may be the same or different, and they are each independently selected from the group consisting of hydrogen, C₁-C₃ alkyl, C₁-C₃ alkoxy, C₆-C₅ aryl, chlorine, bromine, or iodine. Ideally, R₁ and R₂ in formula (B) may be the same or different, and they are each independently selected from the group consisting of hydrogen, methyl, ethyl, propyl, isopropyl, or phenyl. More ideally, Formula (B) compound is maleic anhydride.

In some embodiments, the collagen includes at least one selected from the group consisting of collagen extracted from animal tissues and recombinant collagen. For example, the collagen can be collagen extracted from cowhide and tendon, or the recombinant collagen prepared by using yeast as a genetic engineering carrier.

In some embodiments, the collagen is in a solid and/or liquid state. For example, the collagen can be not only a collagen solution (liquid state) dissolved in a solvent, but also a freeze-dried sponge (solid state), as well as a fibrous collagen slurry (solid-liquid mixture).

In some embodiments, the raw material solution I is obtained by diluting collagen into a solvent. The solvent is selected from at least one of organic acid and inorganic acid. Therefore, in some embodiments, the raw material solution I further includes organic acid and/or inorganic acid. Preferably, the organic acid includes at least one of acetic acid and citric acid, and the inorganic acid preferably includes at least one of hydrochloric acid, sulfuric acid and nitric acid.

In some embodiments, the raw material solution II further includes an organic solvent. The organic solvent preferably includes C₂-C₆ ketone compounds, more preferably acetone.

In some embodiments, the concentration of the collagen in the raw material solution I is 2-20 mg/g. Since collagen has viscosity after dissolution, if the concentration is more than 20 mg/g, the viscosity of the solution will be too high, resulting in poor workability. If the concentration is below 2 mg/g, it will be difficult to form a gel because there are few collagen molecules.

In some embodiments, the concentration of the compound of Formula (B) in the raw material solution II is 0.1-5M. If the concentration is lower than 0.1M, excess organic solvents such as acetone will excessively dilute the collagen solution, which will make it difficult to perform subsequent experiments: on the other hand, if the concentration is higher than 5M, the compound of Formula (B) such as maleic anhydride cannot be completely dissolved, which will affect the quality of the final product.

In some embodiments, the weight ratio of the compound of Formula (B) in the raw material solution II to the collagen in the raw material solution I is (10-30):100. In the present invention, when the weight ratio of the collagen in the raw material solution I to the compound of Formula (B) in the raw material solution II is within this range, a good modified grafting rate can be obtained, and the physical strength of the collagen hydrogel is more excellent. If the ratio is lower than 10:100, the modified grafting rate will become poor, and the collagen hydrogel cannot be formed well, its physical strength performance is weakened, and its practical applicability is not good. On the other hand, even if the amount of the compound of Formula (B) is further increased, that is, when the ratio is higher than 30:100, no better effect can be obtained, and the burden on the cost will be increased instead.

In some embodiments, the reaction temperature of the modification reaction is 0-25° C. For example, the temperature can be 0° C., 2° C., 4° C., 6° C., 8° C., 10° C., 12° C., 15° C., 20° C., 25° C. and any value in between. Ideally, the reaction temperature of the modification reaction is 4-8° C.

In some embodiments, the reaction time of the modification reaction is 8-24 hours. For example, the time can be 8 hours, 12 hours, 16 hours, 20 hours, 24 hours and any value in between. In the present invention, if the modification reaction time falls within this range, the modification can be sufficiently performed. On the contrary, when it is less than 8 hours, sufficient modification cannot be performed. Furthermore, even if the modification time is longer than 24 hours, better results cannot be obtained, but it will increase the burden of time and cost.

In some embodiments, the preparation method further includes performing solid-liquid separation and/or purification treatment on the product after the modification reaction. The solid-liquid separation is ideally carried out by filtration or centrifugation, and more ideally by centrifugation. If centrifugation is used for solid-liquid separation, the obtained solids are compact with lower water content and less consumption: in contrast, the general solid-liquid separation method can easily obtain loose solids with higher water content and higher consumption. The purification treatment is ideally carried out by washing or dialysis, and more ideally by dialysis. If the dialysis method is used to remove impurities, the selectivity of the dialysis membrane to molecular size can be used to diffuse and remove impurities while also retaining macromolecular collagen in the dialysis membrane, thereby obtaining collagen with higher purity.

In a third aspect, the present invention provides a photo-crosslinked biomaterial, which includes a hydrogel formed by curing said modified collagen, or the modified collagen obtained by said method.

In some embodiments, the Young's modulus of the photo-crosslinked biomaterial is 0.02-0.5 kPa, preferably 0.15-0.5 kPa, more preferably 0.3-0.5 kPa. In some embodiments, the bulk modulus of the photo-crosslinked biomaterial is 0.5-20 kPa, preferably 2-20 kPa, more preferably 5-10 kPa. In some embodiments, the yield strength of the photo-crosslinked biomaterial is 10-200 kPa, preferably 25-200 kPa, more preferably 90-150 kPa.

In a fourth aspect, the present invention provides a method for preparing a photo-crosslinked biomaterial, including photocrosslinking and curing the aforementioned modified collagen, or the modified collagen obtained through the aforementioned preparation method with a photoinitiator under light conditions.

In some embodiments, the photoinitiator is 2-hydroxy-4′-(2-hydroxyethoxy)-2-methylpropiophenone (Irgacrue2959) and/or lithium phenyl (2,4,6-trimethylbenzoyl) phosphinate (LAP). Using Irgacure2959 and LAP as the photoinitiator has the advantage of lower cytotoxicity: if other photoinitiators are used, there will be a risk of higher cytotoxicity.

In some embodiments, the use concentration of the 2-hydroxy-4′-(2-hydroxyethoxy)-2-methylpropiophenone is 0.01-1% (w/v) of the overall volume of collagen. If it is less than 0.01%, the gel may not be formed effectively, and if it is higher than 1%, the cytotoxicity may be increased.

In some embodiments, the use concentration of the lithium phenyl (2,4,6-trimethylbenzoyl) phosphinate is 0.01-2% (w/v) of the overall volume of collagen. If it is less than 0.01%, the gel may not be formed effectively, and if it is higher than 2%, the cytotoxicity may be increased.

In some embodiments, the modified collagen is re-suspended in a solvent for photo-crosslinking and curing. The solvent is ideally water or phosphate buffer. In some embodiments, the modified collagen is purified and resuspended in a solvent for photocrosslinking and curing. The amount of 2-hydroxy-4′-(2-hydroxyethoxy)-2-methylpropiophenone is 0.01-1% (w/v) of the volume of the purified collagen solution; and/or the amount of lithium phenyl (2,4,6-trimethylbenzoyl) phosphinate is 0.01-2% (w/v) of the volume of the purified collagen solution.

In some embodiments, the ultraviolet light is used for photocrosslinking and curing.

In some embodiments, the ultraviolet light has a wavelength of 365-405 mm. If ultraviolet light in this range is used, the photoinitiator can absorb it more effectively.

In some embodiments, the irradiation time of the ultraviolet light is more than 15 seconds. Preferably, it is 15-300 seconds. For example, it can be 15 seconds, 30 seconds, 50 seconds, 100 seconds, 150 seconds, 200 seconds, 250 seconds and any value in between. In the present invention, the irradiation time of ultraviolet light can fall within the above range. Illumination can be stopped when the collagen is visually observed to be gelled and has no fluidity. Even if the illumination time lasts longer, it will not get better results, but it will increase the time cost and the risk of unexpected situations. Conversely, if the time is less than 15 seconds, the gel cannot be formed efficiently.

In some embodiments, the compound of Formula (B) such as maleic anhydride can modify the collagen. If the modification is carried out with a commonly known acid instead of an acid anhydride, there may be a possibility that the modification cannot be carried out smoothly: in addition, in terms of acid anhydrides, if other known acid anhydrides such as succinic anhydride are used for modification, the modified collagen has poor reactivity and cannot be gelled under ultraviolet light. Furthermore, the inventors found that if the cross-linking and curing are carried out via ultraviolet light, no cross-linking agent needs to be added, which has the advantages of fast curing, simple preparation process, and less toxic residues, as well as achieving in-situ cross-linking, which solves the defects of traditional hydrogel preparation.

In a fifth aspect, the present invention provides an application of photo-crosslinked biomaterials, which is to apply the aforementioned photo-crosslinked biomaterials or the photo-crosslinked biomaterials s obtained through the aforementioned methods, to the fields of repair and reconstruction of tissues and organs in vivo or in vitro, tissue engineering, 3D printing substrates, or cell culture carriers. Compared with prior art, the present invention has following advantage:
(1) Since collagen is a thermosensitive substance, if the temperature during the production process is too high, its triple helix structure will be destroyed and its original efficacy will lost. However, the process of the present invention does not need to work in a high-temperature environment like the commonly used processes, so the triple helix structure in the collagen can be avoided from being damaged and the integrity of the collagen can be preserved.
(2) The collagen hydrogel prepared according to the present invention has good biocompatibility because no chemical cross-linking agent needs to be added in the manufacturing process, and there is no residual toxicity.
(3) The method for preparing the collagen hydrogel of the present invention is photocrosslinking and curing. The method has short curing time, low cost, and can achieve in-situ crosslinking, thus having wider applicability.
(4) According to the preparation method of the present invention, a collagen hydrogel with excellent physical strength can be prepared with a very high grafting rate.

In addition, the in-situ cross-linked hydrogel of the present invention also has the following advantages:

Traditional hydrogels are applied to the affected area after being gelled. Since its shape is fixed and cannot be adjusted according to the appearance of the affected area, it is difficult to tightly bond with the surrounding tissues, and therefore its application is relatively limited. In contrast, since in situ cross-linked hydrogels generally have no fixed shape, when injected in a relatively low-viscosity state, their physical form changes and forms a hydrogel that is fixed in the interstitial space, which is more tightly bonded to biological tissues such as affected areas than conventional hydrogels.
In situ cross-linked hydrogels have the following advantages for applications in the biomedical field: during the operation, the hydrogel is injected into the affected area and then cross-linked and cured, which is simple and less invasive; in the field of tissue engineering, the cross-linked hydrogel will closely fit the shape of the affected area, which can effectively promote tissue reconstruction: in wound repair applications, in situ cross-linked hydrogels can heal wounds and have the potential to replace traditional suture surgery. Other applications include drug delivery, 3D printing, and gene delivery.

Unless otherwise defined, all technical and scientific terms used herein have the same meanings as commonly used in the field to which this invention belongs. For the purpose of interpreting the present specification, the following definitions will apply and where appropriate, terms used in the singular will also include the plural and vice versa.

The term “collagen” as used herein refers to one of protein families. At least 30 coding genes of collagen chains have been discovered so far, which can form more than sixteen kinds of collagen molecules. According to the structure, it can be divided into fibrous collagen, basement membrane collagen, microfibrillar collagen, collagen anchor, collagen with hexagonal network, non-fibrous collagen, transmembrane collagen, etc. Collagen has good biocompatibility, biodegradability and bioactivity. The collagen used herein can be collagen extracted from animal tissues, such as the collagen extracted from cowhide and tendon: it can also be “recombinant collagen”, such as the recombinant collagen prepared by using yeast as a genetic engineering carrier.

The following examples and figures are provided to help understand the present invention. However, it should be understood that they are only used to illustrate the present invention and do not constitute any limitation. The actual protection scope of the present invention is set forth in the claim set. It should also be understood that any modifications and changes can be made without departing from the spirit of the invention.

### BRIEF DESCRIPTION OF THE DRAWINGS

FIG. 1 shows a flowchart of the preparation method of photo-crosslinked biomaterials according to some embodiments of the present invention;

FIG. 2 depicts a schematic structure diagram of a maleic anhydride-modified collagen (hereinafter, sometimes referred to as ColMe) prepared according to the present invention;

FIG. 3 depicts a schematic structure diagram of a succinic anhydride-modified collagen prepared in the comparative example 1 of the present invention:

FIG. 4 depicts a FT-IR spectrum of ColMe prepared in Example 1 of the present invention;

FIG. 5 depicts the cytocompatibility result of ColMe prepared in Example 1 of the present invention:

FIG. 6 depicts the evaluation of grafting ratio after modification based on different addition amounts of maleic anhydride; and

FIG. 7 shows pictures of the physical strength of Example 1, Example 4 of the present invention and the commercially available GelMA.

### DETAILED DESCRIPTION OF THE INVENTION

In order to make the object, technical solution and advantages of the present invention clearer, the present invention will be further described in detail below in conjunction with examples. The specific embodiments described here are only used to explain the present invention, and are not intended to constitute any limitation to the present invention. Furthermore, in the following description, descriptions of conventional structures and techniques are omitted to avoid unnecessarily obscuring the concept of the present invention.

In the present invention, an acid anhydride having a carbon-carbon double bond structure is grafted on the free amino group of collagen to obtain an acid anhydride-modified collagen. Then, the modified collagen is uniformly mixed with a photoinitiator, reacted under light conditions such as ultraviolet light and undergoes crosslinking and curing, thereby preparing a photo-crosslinked biological material gel.

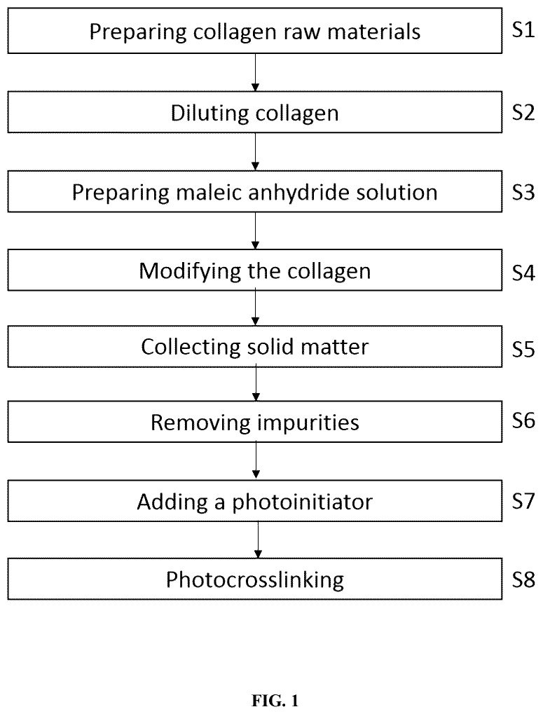

Referring to FIG. 1, which is a flowchart of a method for preparing a photocrosslinkable collagen hydrogel according to an embodiment of the present invention. As shown in FIG. 1, the preparation method of the photocrosslinkable collagen hydrogel of the present invention includes the following steps: providing collagen raw materials (S1); diluting the collagen to obtain raw material solution I (S2); preparing a maleic anhydride solution to obtain raw material solution II (S3): modifying the collagen (S4): collecting solid matter (S5); removing impurities (S6): adding a photoinitiator (S7); and photocrosslinking (S8).

In step (S1), the provided collagen raw materials can use collagen extracted from animal tissues, as well as recombinant collagen. For example, the collagen extracted from cowhide and beef tendon, or recombinant collagen produced by utilizing yeast as a genetic engineering carrier.

In one embodiment, the collagen raw materials are in a liquid form, but it can also be in a solid form or a mixed state of solid and liquid. For example, it can be a collagen solution (liquid) dissolved in a solvent, a freeze-dried sponge (solid), or a fibrous collagen slurry (solid-liquid mixture).

In step (S2), the solvent used to dilute the above-mentioned collagen raw materials includes but not limited to organic acids, inorganic acids, or a combination thereof, such as acetic acid, citric acid, hydrochloric acid, etc. In the raw material solution I, the concentration of the diluted collagen raw materials is 2-20 mg/g. Since collagen has viscosity after dissolution, if the concentration is over 20 mg/g, the viscosity of the solution will be too high, and thereby leading to poor operability. Besides, if the concentration is less than 2 mg/g, it would be difficult to form gel due to few collagen molecules.

In step (S3), the solvent used in the preparation of the maleic anhydride solution is an organic solvent, preferably acetone. Water is a very common solvent, but the acid anhydride is easily decomposed into acid in water, and therefore water is not suitable as a solvent. In the raw material solution II, the concentration of the prepared maleic anhydride solution is 0.1 to 5M. If the concentration is lower than the lower limit, excess acetone may overly dilute the collagen solution, which causing difficulties in subsequent experiments. On the other hand, if the concentration exceeds the upper limit, then the maleic anhydride may not be completely dissolved, which may affect the quality of the final product.

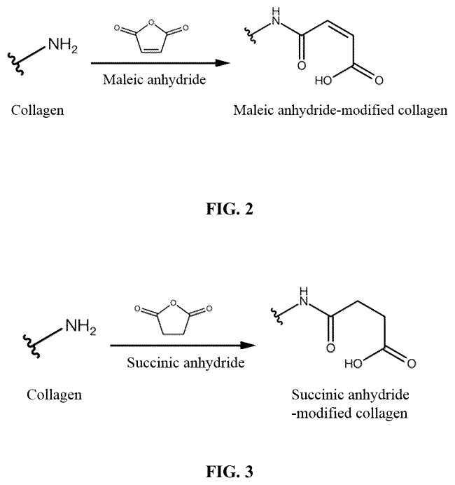

In step (S4), maleic anhydride is used to modify collagen, as shown in FIG. 2. If the acid anhydride is not used but a common acid is used for modification, the modification may not proceed smoothly. Moreover, in terms of acid anhydride, if the commonly known succinic anhydride is used for modification, it can effectively reduce the isoelectric point of collagen, so that the collagen can be dissolved in a neutral environment. However, the structure of succinic anhydride lacks a double bond compared to maleic anhydride, so the modified collagen is less reactive and cannot be gelled under the action of ultraviolet light.

The addition amount of the maleic anhydride solution is to be 10% to 30% by weight relative to the collagen raw materials (W_{maleic} anhydride/W_{collagen} raw materials). If the addition amount falls within this range, a good grafting ratio after modification can be obtained, and the resultant collagen hydrogel has good physical strength. If the addition amount is less than 10%, the grafting ratio after modification is poor, and the collagen hydrogel cannot be formed well, which has poor physical strength, as well as having poor practical applicability. On the other hand, even if the addition amount is higher than 30%, no better results can be obtained, which only increases the burden on the cost.

The time for modifying said diluted collagen raw materials with the maleic anhydride solution is 8 to 24 hours. When the modification time falls within this range, the modification can be fully carried out. If the modification time is less than 8 hours, it will not be able to be fully modified. Furthermore, even if the modification time exceeds 24 hours, no better results can be obtained, which only increases the burden on the time cost.

In step (S5), conventional methods with filtering effect can be used to collect the modified solid matter, which include but not limited to collecting the modified solid matter by centrifugation, strainer (filter collection), or a combination thereof. Among them, the method of collecting the modified solid matter by centrifugation is particularly ideal. By centrifugation, the obtained solid matter is compact, with lower water content and consumption. On the other hand, the conventional collection method may easily obtain looser solid matter, with higher water content and consumption.

In step (S6), the method for removing impurities can be dialysis or washing with liquid. Among them, when removing impurities, the dialysis method can take advantage of the molecular size selectivity of the dialysis membrane, so that while the impurities are diffused and removed, the macromolecule collagen is also trapped in the dialysis membrane, thereby obtaining collagen with higher purity. Therefore, the dialysis method is ideal.

In step (S7), 2-hydroxy-4′-(2-hydroxyethoxy)-2-methylpropiophenone (hereinafter called Irgacure 2959, manufactured by Sigma-Aldrich) or lithium phenyl-2,4,6-trimethylbenzoylphosphinate (hereinafter called LAP, manufactured by Sigma-Aldrich) can be used as a photoinitiator. Using Irgacure 2959 and LAP as photoinitiators has the advantage of lower cytotoxicity: on the contrary, if other photoinitiators are used, the cytotoxicity will be higher, which is not ideal. When using Irgacure 2959, the usage amount is 0.01 to 1% (w/v) of the volume of collagen solution after removing the impurities. The gel cannot be effectively formed if the usage amount is less than 0.01%, and if the amount is more than 1%, there is a concern about excessive cytotoxicity. When using Irgacure 2959, the usage amount is 0.01 to 2% (w/v) of the volume of collagen solution after removing the impurities. The gel cannot be effectively formed if the usage amount is less than 0.01%, and if the amount is more than 2%, there is a concern about excessive cytotoxicity.

In step (S8), ultraviolet light is used as the light source for photocrosslinking and curing, and its wavelength is 365 nm˜405 nm. If the wavelength is within this range, the photoinitiator can perform effective absorption. After UV light is irradiated for more than 15 seconds (e.g., between 15 seconds and 300 seconds), the light can be stopped when the collagen is glued and has no fluidity by visual observation. Even if the light is continued for a longer period of time, no better results can be obtained, which only increases the burden on the time cost, and may cause unexpected situations. Conversely, if the illumination time is less than 15 seconds, the glue cannot be effectively formed.

The following examples are used to illustrate the present invention in detail. However, these examples are for illustrative purposes only, and it does not mean that the present invention can only be limited to the aspects of these examples.

### Example 1

A process of preparing a photocrosslinkable collagen hydrogel, the specific steps are as follows:
(1) the bovine-derived collagen raw materials in the form of a lyophilized sponge were diluted with acetic acid to a concentration of 8 mg/g.
(2) acetone was used as a solvent to prepare 2.5 M maleic anhydride solution.
(3) the maleic anhydride solution obtained in step (2) was added in a manner of 30% by weight relative to the collagen raw materials (W_{maleic} anhydride/W_{collagen}).
(4) the modification was carried out for 24 hours, and the working temperature is 4° C.
(5) the solid matter was centrifuged and collected.
(6) a phosphate buffer solution was used as an external fluid for dialysis, where the molecular weight cut-off of the dialysis membrane is 12-14 kDa.
(7) adding LAP as a photoinitiator at a concentration of 0.06% (w/v) of the volume of collagen solution redissolved in water after dialysis.
(8) after irradiating with ultraviolet light with a wavelength of 365 nm for 60 seconds, a transparent and uniform maleic anhydride-modified collagen hydrogel (hereinafter, sometimes referred to as ColMe) was obtained.

### Example 2

A process of preparing a photocrosslinkable collagen hydrogel, the specific steps are as follows:
(1) the bovine-derived collagen raw materials in the form of a lyophilized sponge were diluted with acetic acid to a concentration of 20 mg/g.
(2) acetone was used as a solvent to prepare 5 M maleic anhydride solution.
(3) the maleic anhydride solution obtained in step (2) was added in a manner of 18% by weight relative to the collagen raw materials (W_{maleic} anhydride/W_{collagen}).
(4) the modification was carried out for 8 hours, and the working temperature is 4° C.
(5) the solid matter was centrifuged and collected.
(6) a phosphate buffer solution was used as an external fluid for dialysis, where the molecular weight cut-off of the dialysis membrane is 12-14 kDa.
(7) adding LAP as a photoinitiator at a concentration of 2% (w/v) of the volume of collagen solution redissolved in water after dialysis.
(8) after irradiating with ultraviolet light with a wavelength of 365 nm for 15 seconds, a transparent and uniform ColMe hydrogel was obtained.

### Example 3

A process of preparing a photocrosslinkable collagen hydrogel, the specific steps are as follows:
(1) the bovine-derived collagen raw materials in the form of a lyophilized sponge were diluted with acetic acid to a concentration of 2 mg/g.
(2) acetone was used as a solvent to prepare 0.1 M maleic anhydride solution.
(3) the maleic anhydride solution obtained in step (2) was added in a manner of 10% by weight relative to the collagen raw materials (W_{maleic} anhydride/W_{collagen}).
(4) the modification was carried out for 24 hours, and the working temperature is 4° C.
(5) the solid matter was centrifuged and collected.
(6) a phosphate buffer solution was used as an external fluid for dialysis, where the molecular weight cut-off of the dialysis membrane is 12-14 kDa.
(7) adding Irgacrue2959 as a photoinitiator at a concentration of 0.1% (w/v) of the volume of collagen solution redissolved in water after dialysis.
(8) after irradiating with ultraviolet light with a wavelength of 365 nm for 300 seconds, a transparent and uniform ColMe hydrogel was obtained.

### Example 4

The collagen hydrogel was prepared by the same method as in Example 1, except that the addition amount of maleic anhydride was changed to 9% (W_{maleic} anhydride/W_{collagen}).

### Comparative Example 1

Referring to the schematic structure diagram in FIG. 3, the collagen hydrogel was prepared by the same method as in Example 1, except that the maleic anhydride was changed to succinic anhydride. The results are shown in Table 1.

TABLE 1

| | Example 1 | Comparative example 1 |
|---|---|---|
| Form gel | YES | NO |

### <FT-IR Test of Maleic Anhydride-Modified Collagen (ColMe) Hydrogel Cells>

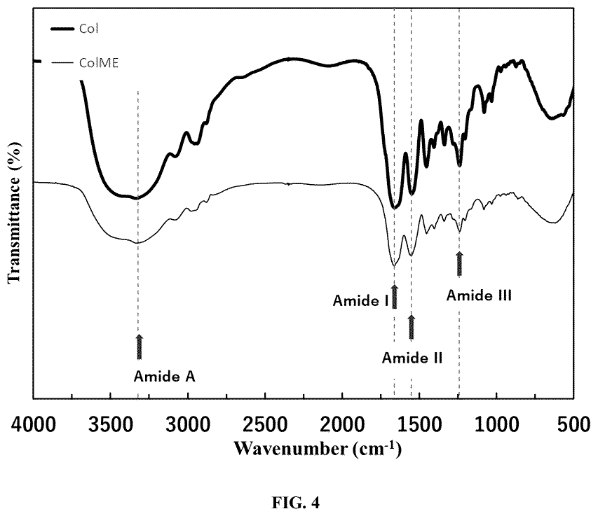

Referring to FIG. 4, the photocrosslinkable collagen hydrogel obtained in Example 1 was subjected to FT-IR measurement. Comparing collagen (Col) and modified collagen (ColMe), both show the characteristic peaks of collagen, including 3370 cm⁻¹ (amide A), 1654 cm⁻¹ (amide I), 1554 cm⁻¹ (amide II) and 1241 cm⁻¹ (amide III). The absorption value ratio (AIII/A1450) of the characteristic peak of amide III and the characteristic peak of 1450 cm⁻¹ was further compared, as the basis for the integrity of the triple helix structure of collagen, and it was confirmed that the maleic anhydride-modified collagen completely preserved the structural characteristics of collagen.

### <To Evaluate the Compatibility of Maleic Anhydride-Modified Collagen (ColMe) Hydrogel Cells>

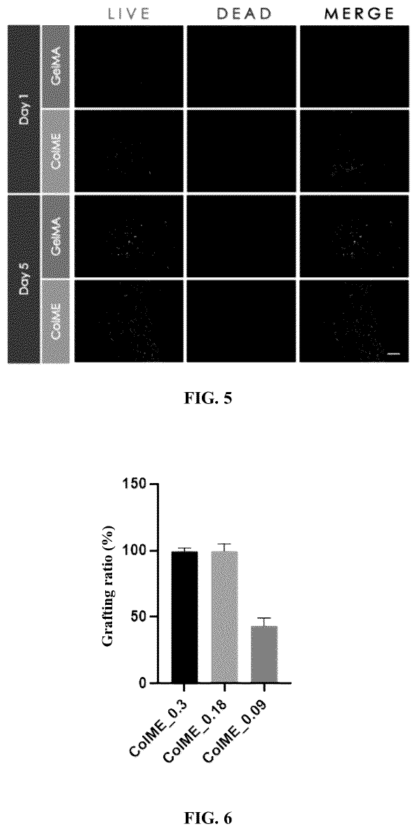

The in vitro cell compatibility of the ColMe hydrogel prepared in Example 1 was tested and compared with the commercially available gelatin methacryloyl (GelMA). The results showed that ColMe hydrogel is non-cytotoxic to L929 cells (FIG. 5), and has good cell compatibility like commercially available GelMA.

### <To Evaluate Grafting Ratio>

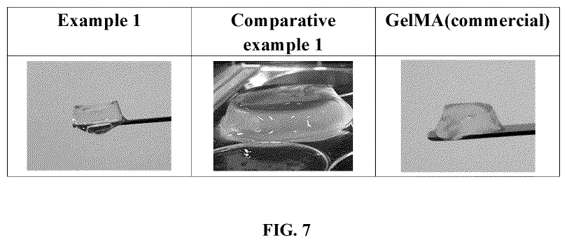

Trinitrobenzenesulfonic acid assay (TNBS assay) was used to detect the grafting rate of different addition amounts of maleic anhydride after modification for Example 1, Example 2 and Example 4 (It is represented by ColMe_0.3, ColME_0.18, and ColMe_0.09 below respectively). The results showed that both ColMe 0.3 and ColME_0.18 had a grafting ratio of 99%. In contrast, ColMe_0.09 only had a grafting ratio of 43% (FIG. 6). In addition, the calculation method of grafting ratio and the information of each group are as follows.
grafting ratio:

$$( 1 - \frac{Free amino groups concentration of collagen after modification}{F ree amino groups concentration of collagen before modification} ) * 100 \%$$

$$ColME\_ 0.3 : {W}_{maleic anhydride} / {W}_{collagen} = 30 \% ( Example 1 )$$

$$ColME\_ 0.18 : {W}_{maleic anhydride} / {W}_{collagen} = 18 \% ( Example 2 )$$

$$ColME\_ 0.09 : {W}_{maleic anhydride} / {W}_{collagen} = 9 \% ( Example 4 )$$

### <Physical Strength Assessment>

For the GelMA of Example 1, Example 4, and the commercially available product, the physical strength was measured. A rheometer was used to detect Young's modulus, Bulk modulus and yield strength. The results are shown in Table 2.

Moreover, in order to better understand the physical strength of GelMA of Example 1, Example 4, and the commercially available product, their pictures were also shown in FIG. 7.

TABLE 2

| | Example 1 | Example 4 | GelMA(commercial) |
|---|---|---|---|
| Young's modulus (kPa) | 0.310 | 0.032 | 0.119 |
| Bulk modulus (kPa) | 8.805 | 0.903 | 1.179 |
| Yield strength (kPa) | 94.89 | 10.46 | 20.25 |

According to the results in Table 2 and FIG. 7, it could be found that the photo-crosslinked collagen hydrogel prepared by satisfying the process conditions of the present invention has more sufficient physical strength. Therefore, it can achieve better physical support in the field of tissue engineering, and it is difficult to make the hydrogel damage and displacement by external forces after the gel is formed, so as to perform better hydrogel functions, such as drug release and cell adhesion, promoting the repair of surrounding tissues, etc.

Although the present invention has been specifically shown and described with reference to exemplary embodiments, it will be understood by those of ordinary skill in the art to which the invention pertains that various changes in form and details may be made to the embodiments of the present invention without departing from the spirit and scope of the present invention as defined in the following claims and their equivalents.

1. A modified collagen comprising a collagen grafted with a group represented by Formula (A): 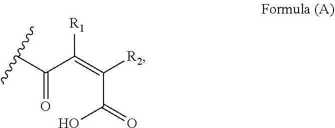
wherein R₁ and R₂ in the Formula (A) are the same or different, and are each independently selected from the group consisting of hydrogen, C₁-C₆ alkyl, C₁-C₆ alkoxy, C₆-C₁₀ aryl, or halogen, and wherein [symbol] represents the position where the group represented by Formula (A) connected to the collagen.

2. The modified collagen according to claim 1, wherein the group represented by the Formula (A) is grafted on a free amino group of the collagen.

3. The modified collagen according to claim 2, wherein the grafting rate of the modified collagen is at least 40%.

4. A method for preparing a modified collagen, comprising carrying out a modification reaction between a collagen-containing raw material solution I and a raw material solution II with a compound of Formula (B), 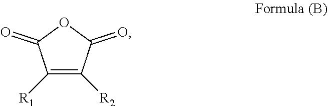
wherein R₁ and R₂ in the Formula (B) are the same or different, and are each independently selected from the group consisting of hydrogen, C₁-C₆ alkyl, C₁-C₆ alkoxy, C₆-C₁₀ aryl, or halogen.

5. The method according to claim 4, wherein the collagen comprises at least one selected from the group consisting of collagen extracted from animal tissues and recombinant collagen, and the collagen is in a solid and/or liquid state.

6. The method according to claim 5, wherein the collagen comprises a collagen extracted from cowhide and tendon, or a recombinant collagen prepared by using yeast as a genetic engineering carrier.

7. The method according to claim 4, wherein the raw material solution I at least meets any of the following conditions:
the concentration of the collagen is 2-20 mg/g,
comprising organic and/or inorganic acids;
and the raw material solution II at least meets any of the following conditions:
the concentration of the Formula (B) compound is 0.1-5M,
the weight ratio of the Formula (B) compound to the collagen in the raw material solution I is 10-30:100,
comprising an organic solvent.

8. The method according to claim 7, wherein the organic acid comprises at least one of acetic acid and citric acid, and/or wherein the inorganic acid comprises at least one of hydrochloric acid, sulfuric acid and nitric acid.

9. The method according to claim 7, wherein the organic solvent comprises C₂-C₆ ketone compounds.

10. The method according to claim 9, wherein the organic solvent comprises acetone.

11. The method according to claim 4, further satisfying at least one of the following conditions:
the reaction time of the modification reaction is 8-24 hours,
the reaction temperature of the modification reaction is 0-25° C., and
further comprises performing solid-liquid separation and/or purification treatment on the product after the modification reaction.

12. The method according to claim 11, wherein the reaction temperature of the modification reaction is 4-8° C.

13. The method according to claim 1, wherein the solid-liquid separation is performed by filtration or centrifugation.

14. The method according to claim 11, wherein the purification treatment is performed by washing or dialysis.

15. A photo-crosslinked biomaterial, comprising a hydrogel formed by curing the modified collagen as in claim 1.

16. The photo-crosslinked biomaterial according to claim 15, further satisfying at least one of the following (a)-(c):
(a) the Young's modulus of the photo-crosslinked biomaterial is 0.02-0.5 kPa;
(b) the bulk modulus of the photo-crosslinked biomaterial is 0.5-20 kPa;
(c) the yield strength of the photo-crosslinked biomaterial is 10-200 kPa.

17. The photo-crosslinked biomaterial according to claim 15, further satisfying at least one of the following (a)-(c):
(a) the Young's modulus of the photo-crosslinked biomaterial is 0.3-0.5 kPa;
(b) the bulk modulus of the photo-crosslinked biomaterial is 5-10 kPa;
(c) the yield strength of the photo-crosslinked biomaterial is 90-150 kPa.

18. A method for preparing a photo-crosslinked biomaterial, comprising photocrosslinking and curing the modified collagen as in claim 1.

19. The method according to claim 18, wherein the photoinitiator is 2-hydroxy-4′-(2-hydroxyethoxy)-2-methylpropiophenone and/or lithium phenyl-2,4,6-trimethylbenzoylphosphinate.

20. The method according to claim 19, wherein the amount of 2-hydroxy-4′-(2-hydroxyethoxy)-2-methylpropiophenone is 0.01-1% (w/v) and/or the amount of lithium phenyl (2,4,6-trimethylbenzoyl) phosphinate is 0.01-2% (w/v).

21. The method according to claim 18, wherein ultraviolet light is used for photocrosslinking and curing.

22. The method according to claim 21, further satisfying at least one of the following:
the wavelength of the ultraviolet light is in a range of 365-405 mm, and
the irradiation time of the ultraviolet light is at least 15 seconds.

23. An application of photo-crosslinked biomaterials, which is to apply the photo-crosslinked biomaterials of claim 15 or the photo-crosslinked biomaterials obtained through the method of claim of 18, to the fields of repair and reconstruction of tissues and organs, tissue engineering, 3D printing substrates, or cell culture carriers.
